(19) United States
(12) Patent Application Publication
(10) Pub. No.: US 2017/0156640 A1
(43) Pub. Date: Jun. 8, 2017

(54) SYSTEM AND METHOD FOR DETECTION AND MEASUREMENT OF BODY PART MOVEMENT USING CAPACITIVE SENSORS AND INERTIAL SENSING SYSTEMS

(71) Applicants: Ryan ROBUCCI, Halethorpe, MD (US); Nilanjan BANERJEE, Columbia, MD (US)

(72) Inventors: Ryan ROBUCCI, Halethorpe, MD (US); Nilanjan BANERJEE, Columbia, MD (US)

(21) Appl. No.: 15/319,688

(22) PCT Filed: Jun. 18, 2015
(86) PCT No.: PCT/US2015/036495

§ 371 Date: Dec. 16, 2016

Related U.S. Application Data
(63) Continuation-in-part of application No. 14/523,347, filed on Oct. 24, 2014.
(60) Provisional application No. 62/013,593, filed on Jun. 18, 2014.
(60) Provisional application No. 61/894,987, filed on Oct. 24, 2013.

(51) Int. Cl. A61B 5/11; H05K 1/03; A61B 5/00

(52) U.S. Cl. CPC A61B 5/1118; A61B 5/6828; A61B 5/7278; A61B 5/4595; A61B 5/4815; A61B 5/6831; H05K 1/038; A61B 2562/0219; H05K 2201/10151

(57) ABSTRACT

Systems and methods for distinctly detecting the movement of a first object and the movement of a second object relative to the first object are provided that utilize a capacitive sensor array and an inertial sensing system. The systems and methods are particularly suited for the detection and measurement of first body part movement (e.g., leg movement) and second body part movement (e.g., foot flexion). In one embodiment, the system utilizes a textile-based capacitive sensor array and one or more inertial sensors disposed on a flexible structure that is adapted to be wrapped around a body part, such as a leg. The systems and methods can be used to calculate periodic leg movements (PLM) and general leg movements (GLM) based on detected movements of the leg and foot.

This application is a continuation-in-part of U.S. patent application Ser. No. 14/523,347 filed Oct. 24, 2014, which claims priority to U.S. Provisional Application Ser. No. 61/894,987 filed Oct. 24, 2013. This application also claims priority to U.S. Provisional Application Ser. No. 62/013,593 filed Jun. 18, 2014. The entire disclosures of the above-identified applications are incorporated herein by reference.

### BACKGROUND OF THE INVENTION

1. Field of the Invention

The present invention relates generally to position and movement detection and measurement and, more specifically, the simultaneous and distinct detection and measurement of the movement of a first object, as well as the movement of second object relative to the first object, utilizing capacitive sensing and inertial sensing.

2. Background of the Related Art

The Background of the Related Art and the Detailed Description of Preferred Embodiments below cite numerous technical references, which are listed in the Appendix below. The numbers shown in brackets (“[ ]”) refer to specific references listed in the Appendix. For example, “[1]” refers to reference “1” in the Appendix below. All of the references listed in the Appendix below are incorporated by reference herein in their entirety.

Leg movements (LM) during sleep and rest (LMS) have significant implications for health, quality of life and sleep, and they can be evaluated for an individual at home during normal sleep. Leg movements have long been recognized in sleep medicine for their significance to sleep and are routinely measured in clinical sleep laboratory studies. However, both the high nightly variability (requiring multiple nights of data for reliable individual LMS assessment [1, 2]) and the lack of analytics for fully identifying and characterizing LMS has severely limited their clinical utility. The lack of accessible, affordable, and user-friendly systems for identifying and measuring clinically significant LMS over multiple nights has been a major barrier to clinical evaluation of LMS.

The clinical significance of rest and sleep related leg movement depends on the movement type, i.e., classical periodic leg movements (PLM) and all general leg movements (GLM) of rest and sleep.

Significance of General Leg Movements (GLM) of Rest and Sleep

Significance of General Leg Movements of Rest and Sleep (GLM):

GLM, unlike PLM, are not well studied, but they provide a direct measure of restless sleep and are likely more significant than arm movements. Changing sleep position to get comfortable and rotating and extending the leg repeatedly during the sleep period reflect the common clinical report of the restless insomnia patient. These reports rarely include arm movements.

Movement is arousing and inactivity is needed for sleep. In humans, this is particularly true given the elaborate brain and spinal systems activated to support motor control and alertness to stimuli for safe, effective upright movement on two legs. However, due to a lack of enabling technology, epidemiological and clinical studies have not generally been able to objectively identify the insomnia phenotype of excessive movement or restlessness.

Measuring GLM provides an objective assessment of disrupted sleep onset or maintenance relevant for defining restless sleep. Moreover, assessing the GLM that are not PLM may potentially support evaluating circadian rhythm disturbances and estimating sleep time and quality. Thus, GLM assessment enables identification of insomnia defined by restlessness at sleep onset and during sleep. This type of insomnia needs to be better evaluated.

It is unclear from current data whether it is all GLM of significant amplitude or only those related or unrelated to PLM that are more significant for restless sleep, but it is likely that the overall amplitude of all GLM represents restlessness in sleep. It also seems likely that the degree of GLM and its relation to PLMS may define different insomnia phenotypes to guide treatment and research development.

Measuring GLM timing, amount, amplitude and PLM-relation would aid diagnosis and phenotype considerations. Continued monitoring would support treatment, evaluations, and improve the at-home health behaviors of the user. Thus, inexpensive, easily used and repeated GLM home monitoring would both benefit the insomnia patient and enable insomnia research. Accordingly, there is a significant commercial market for home GLM measurements, particularly if developed with guided general treatment options for the 25 million insomnia sufferers in the United States alone.

Significance of Period Leg Movements During Sleep and Resting

PLM during sleep (PLMS), unlike GLM, have been well defined and extensively studied. PLM are defined by anterior tibialis activation, presently measured by an electromyograph (EMG), producing foot flexion but not necessarily leg movement. As many as 50% of PLMS may occur without significant leg movement, thus leg activity monitoring is inadequate for PLMS evaluation [3]. There was an initial view that PLMS occurred mostly with restless legs syndrome (RLS) and that they disrupted sleep producing the periodic limb movement disorder of sleep. Studies found PLMS do not per se disrupt sleep [4, 5] and that they occur at moderately high rates in older healthy adults [6]. This led to some uncertainly about their clinical significance [7], but these studies had no assessment of amplitude of the PLMS or relation to the degree of any associated leg movement (GLM).

PLMS foot flexion only without significant GLM may not impact sleep. Conversely, PLMS with significant leg movement (GLM) may be associated with significant sleep disruption and possibly more extreme autonomic arousal seen with the PLMS. Moreover, it has recently been discovered that PLMS are less significant with regards to sleep disruption, but are nevertheless still generally significant because they represent periodic physiological processes that have health significance and also indicate disease state [5].

The PLMS produce, with each movement, significant transient blood pressure and heart rate increases [8, 9]. This does not occur for actual non-PLMS leg movements even with equivalent or greater amplitude [8]. Moreover, as might be expected given these related events, frequent PLMS are associated with left ventricular hypertrophy, poor cardiovascular outcomes [10] [11, 12] and future development of atrial fibrillation [11]. The actual level of PLMS associated with these conditions remains uncertain, and whether treatments reducing PLMS will reduce cardiac morbidity requires future studies [13].

PLMS have particular significance for restless legs syndrome (RLS). They occur when recorded over several nights for 85 to 90+% of all RLS patients [4] [1]. They support the diagnosis of RLS and provide the only objective evaluation of RLS severity [14] [15] and treatment efficacy [16-18]. Thus, this measure is used by clinicians to evaluate RLS patients and also by the patient to follow treatment over time.

PLMS occur in connection with several medical conditions. The characteristic features of PLMS (number, inter-movement interval, autocorrelation, distribution pattern over the night, duration, amplitude) differ for these underlying conditions, e.g., RLS [19], sleep apnea [20], and REM behavior disorder [21] [22].

PLMS also have genetic association shared with RLS on BTBD9, TOX3/BC034767, MEIS1, MAP2K5/SKOR1, and PTPRD [23] that is particularly strong for the allelic variation on BDBT9 [24]. Moreover, higher PLMS/hr rates of older adults occurs mostly for those whose families have RLS [25]. Thus PLMS may indicate shared familial genetic RLS risk and possible susceptibility to environmental factors related to RLS.

Significance of PLMS in Children

PLMS in children are particularly important. PLMS greater than 5 per hour in children is not considered normal [6, 26]. In one study, these occurred in 7.7% of all children evaluated and were associated with increased arousals and poorer sleep [26]. PLMS occur commonly with attention deficit hyperactivity disorder (ADHD) [27, 28] and RLS [29]. In children, the PLMS represent disturbed sleep that exacerbates associated problems, e.g. attention deficit disorder (ADD) and autism.

PLMS in children often occur with low but normal serum ferritin, indicating possible functional CNS iron deficiency. Children with PLMS and low normal serum iron (<50-75 mcg/l) can be successfully treated with oral iron [30, 31]. Blood tests without PLMS is not sufficient to justify putting a child through the discomfort and struggle associated with taking oral iron. Treatment response and how long to continue iron treatment cannot be easily defined by repeated blood tests, but rather by repeated PLMS evaluations. Providing an effective, accessible and inexpensive method for easy initial and continuing evaluation of PLMS opens a door for improved pediatric care. Some ADHD children with PLMS may not only do better after oral iron treatment, but may be able to decrease or discontinue their other medications improving their health and reducing medical costs.

Overall PLMS provide an important indication of possible current and future health issues. It is particularly important to measure PLMS over several nights since there is a high degree of night-to night variability [1] [2], requiring 3 to 5 nights for stable individual measurement. It is also important to have repeated measurements that can be easily and accurately obtained for one person under different behavioral, environmental and medical conditions. An accessible, inexpensive evaluation of PLMS can provide potentially important medical information for a user to evaluate sleep and health, and for doctors to help evaluate patients.

### SUMMARY OF THE INVENTION

An object of the invention is to solve at least the above problems and/or disadvantages and to provide at least the advantages described hereinafter.

Therefore, an object of the present invention is to provide a system and method for distinctly detecting and measuring the movement of a first object, as well as the movement of a second object with respect to the first object.

Another object of the present invention is to provide a system and method for distinctly detecting and measuring the movement of an object, as well as the movement of an appendage of the object with respect to the object.

Another object of the present invention is to provide a system and method for distinctly detecting and measuring the movement of a first body part, as well as the movement of a second body part with respect to the first body part.

Another object of the present invention is to provide a system and method for distinctly detecting and measuring the movement of a body part, as well as the movement of an appendage of the body part with respect to the body part.

Another object of the present invention is to provide a system and method for distinctly detecting and measuring the movement of a human leg, as well as the movement of the foot with respect to the human leg.

Another object of the present invention is to provide a multi-sensor measurement system that utilizes one or more inertial sensors and a capacitive sensor array.

Another object of the present invention is to provide a multi-sensor measurement system that utilizes one or more inertial sensors and a flexible capacitive sensor array.

Another object of the present invention is to provide a multi-sensor measurement system that utilizes one or more inertial sensors and a textile-based capacitive sensor array.

Another object of the present invention is to provide a multi-sensor measurement system that utilizes an accelerometer and a capacitive sensor array.

Another object of the present invention is to provide a multi-sensor measurement system that utilizes an accelerometer, a gyroscope and a capacitive sensor array.

Another object of the present invention is to provide a multi-sensor measurement system that wraps around an object.

Another object of the present invention is to provide a multi-sensor measurement system that wraps around a body part.

Another object of the present invention is to provide a multi-sensor measurement system that wraps around a leg.

Another object of the present invention is to provide a system and method for detecting periodic leg movements and general leg movements.

Another object of the present invention is to provide a system and method for detecting periodic leg movements during sleep and general leg movements during sleep.

To achieve at least the above objects, in whole or in part, there is provided a multi-sensor measurement system, comprising at least one support structure, a capacitive sensor array disposed on the at least one support structure, wherein the capacitive sensor array comprises at least two conductive plates, and at least one inertial sensor disposed on the at least one support structure.

To achieve at least the above objects, in whole or in part, there is also provided a system for detecting movement of a first body part and movement of a second body part with respect to the first body part, comprising at least one flexible support structure, wherein the at least one flexible support structure is adapted to be wrapped around the first body part, a capacitive sensor array disposed on the at least one flexible support structure, wherein the capacitive sensor array comprises at least two flexible conductive plates, and at least one inertial sensor disposed on the at least one flexible support structure.

To achieve at least the above objects, in whole or in part, there is also provided a method of detecting movement of a first body part and movement of a second body part relative to the first body part, comprising the steps of detecting movement of the first body part based on signals from at least one inertial sensor, and detecting movement of the second body part relative to the first body part based on signals from a capacitive sensor array.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1A is a schematic diagram of a multi-sensor measurement system, in accordance with one preferred embodiment of the present invention;

FIG. 1B is a schematic diagram of a multi-sensor measurement system, in accordance with another preferred embodiment of the present invention;

FIG. 2 is a schematic diagram illustrating one preferred embodiment of the capacitive sensor array assembly, in accordance with one preferred embodiment of the present invention;

FIG. 3 is a schematic diagram of an inertial sensing system, in accordance with one preferred embodiment of the present invention;

FIG. 4A is a schematic diagram of a multi-sensor measurement system, in accordance with another preferred embodiment of the present invention;

FIG. 4B is a schematic diagram of a multi-sensor measurement system, in accordance with another preferred embodiment of the present invention;

FIGS. 5A and 5B are schematic diagrams illustrating the multi-sensor measurement system attached to a leg, in accordance with one preferred embodiment of the present invention;

FIGS. 6A and 6B are perspective views showing multi-sensor measurement system in isolation (FIG. 6A) and wrapped around and secured to a leg (FIG. 6B), in accordance with one preferred embodiment of the present invention;

FIG. 7A are graphs are graphs showing the capacitance measured by six capacitor plates, as well as the acceleration measured by an accelerometer, in accordance with one preferred embodiment of the present invention; and

FIG. 7B are graphs showing the capacitance measured by six capacitor plates, as well as the acceleration measured by an accelerometer for three different sleep positions, in accordance with one preferred embodiment of the present invention.

### DETAILED DESCRIPTION OF PREFERRED EMBODIMENTS

The systems and methods of the present invention are particularly suited for detection and measurement of the movement of body parts (e.g., legs and foot flexion), and thus the systems and methods of the present invention will be predominantly described and illustrated in this context. However, it should be appreciated that the present invention may be utilized in other applications in which one wants to simultaneously and distinctly detect and measure the movement of a first object, as well as the movement of a second object relative to the first object.

The first major technological barrier for leg movement evaluation is separating GLM from PLM, and activity meters fail to do this. PLM and PLMS are defined by activation of the anterior tibialis muscle. This produces a physiological flexion of the foot at the ankle with extension of the big toe.

PLMS can and frequently do occur with essentially no leg movements and only physiological foot flexion. In clinical evaluation of videos of general sleep patients, between 10 and 50% of PLMS occurred without noticeable leg movement. It seems unlikely that these would relate to sleep disturbance, but may still mark significant autonomic arousal and heart rate and blood pressure changes. In another study, 65% of the PLMS occurred without leg movements, while 55% of leg movements detected by an accelerometer on the ankle (GLMS) were not PLMS [3]. Activity meters generally report more leg movements than visual scorers [32] and fail to accurately measure PLMS for insomnia patients who have a highly variable GLM rate falsely reported as PLMS by activity meters [33].

RLS patients represent a special situation where GLMS and PLMS tend to overlap. Thus, leg activity meters have shown modest agreement with sleep lab PLMS for RLS patients but not other patients [33]. Even for RLS, there is a wide range of error (−70% to +120%) largely reflecting the variable degree of GLMS and PLMS overlap [32]. Thus, using activity to measure PLMS is inadequate and only possible in uncertain, unknown and variable conditions where PLMS and GLMS overlap.

The second major technological barrier is evaluating the magnitude of PLM and GLM. Magnitude of the PLM could not previously be adequately analyzed since the EMG used for prior PLMS measurements had surface electrodes with un-calibrated signals. Thus, no prior PLM research or clinical evaluation has been able to accurately assess the amplitude of the movement and this, in part, may account for the lack of relation to sleep disruption.

The third major technological barrier is separating resting/sleep from active/awake movements. Getting up or sitting on the bed and tapping a foot can look like PLMS for recordings without EEG.

The present invention utilizes an inertial sensing system to measure movement of a first body part and proximity-based motion detection using capacitive sensor arrays to measure movement of a second body part relative to the first body part. The use of capacitive sensor arrays for motion detection is described in related co-pending U.S. patent application Ser. No. 14/523,347, which is incorporated by reference herein in its entirety.

In one preferred embodiment, textile-based capacitive sensor arrays are used that are constructed using conductive textile and that can be integrated into other textiles, such as a wearable band that can be worn around the body part being measured, as will be explained in more detail below.

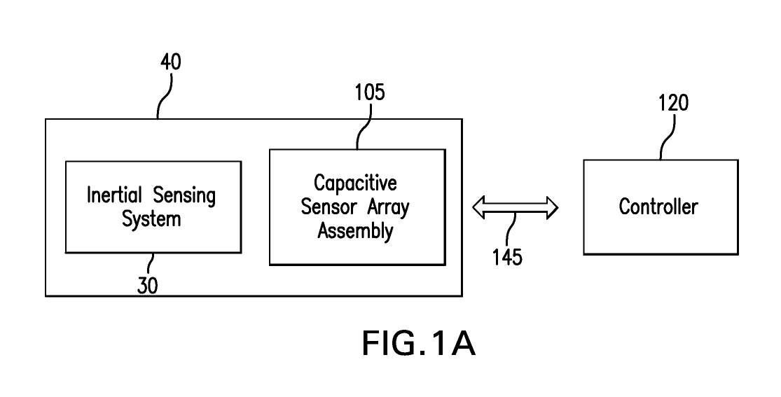

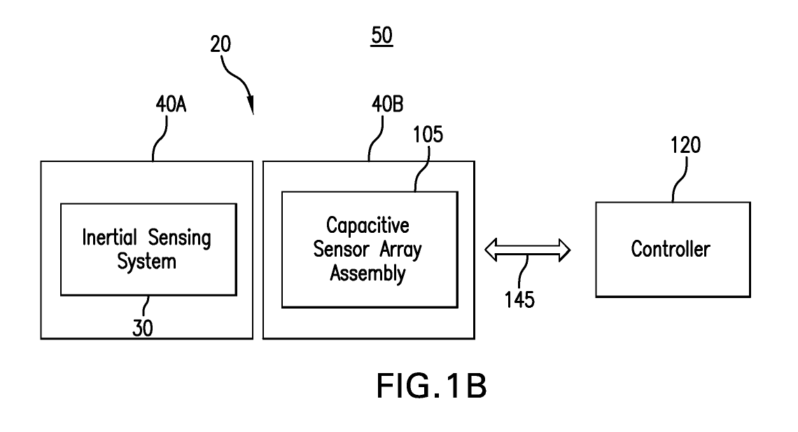

FIG. 1A is a schematic diagram of a multi-sensor measurement system (MSMS) 10, in accordance with one preferred embodiment of the present invention. The MSMS 10 includes a multi-sensor assembly 20 and a controller 120. The multi-sensor assembly 20 includes a capacitive sensor array (CSA) assembly 105 and an inertial sensing system 30.

Communication between the multi-sensor assembly 20 and the controller 120 is enabled via connections 145. Connections 145 can be wired connections, wireless connections, wireless inductive connections and/or capacitive connections using systems and methods well known in the art.

The CSA assembly 105 and inertial sensing system 30 are mounted on or integrated with a support structure 40. Although the support structure 40 is shown as a single structure on which the CSA assembly 105 and inertial sensing system 30 are mounted on or integrated with, it should be appreciated that multiple support structures 40 may be used while still falling within the scope of the present invention. For example, the CSA assembly 105 and initial sensor module 30 could each be mounted on or integrated with its own separate support structure 40, as shown in FIG. 1B.

As shown in the MSMS embodiment 50 of FIG. 1B, the inertial sensing system 30 is mounted on or integrated with support structure 40A and CSA assembly 105 is mounted on or integrated with support structure 40B. Support structures 40, 40A and 40B are preferably flexible structures, suitably flexible fabric, that allows for the multi-sensor assembly 20 to be wrapped around a body part, as will be explained in more detail below. Examples of materials that can be used for the support structure 40 include, but are not limited to elastic bands and cotton ace bandages.

A more detailed explanation of the operation of the MSMS 10 will be provided below in the context of the measurement of PLMS and GLMS, but in general the multi-sensor assembly 20 is mounted on or attached to a first object whose movement is being measured (e.g., a human leg) in close proximity to a second object whose movement is being measured relative to the first object (e.g., the foot). The inertial sensing system 30 is adapted to measure movement of the first object, while the CSA assembly is adapted to measure movement of a second object relative to the first object via capacitive sensing. The inertial sensing system can include any number of inertial sensors. Examples of inertial sensors include, but are not limited to accelerometers and gyroscopes.

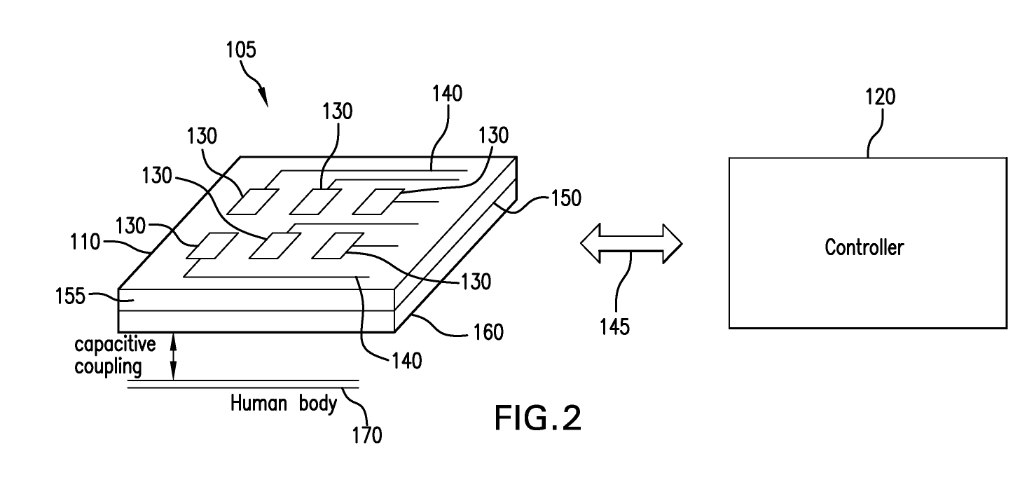

FIG. 2 is a schematic diagram illustrating one preferred embodiment of the CSA assembly 105. A detailed explanation of the operation of the CSA assembly 105 is provided in related co-pending U.S. patent application Ser. No. 14/523,347, which is incorporated by reference herein in its entirety.

The CSA assembly 105 includes a CSA 110 that is made up of at least two capacitor plates 130 (preferably at least six capacitor plates 130) and conductive wires 140 that carry signals from the capacitor plates 130. The controller 120 is in communication with the conductive wires 140 via connections 145, which transfer the signals to the controller 120. As discussed above, connections 145 can be wired connections, wireless connections, wireless inductive connections and/or capacitive connections using systems and methods well known in the art.

The term “capacitor plate” is used to refer to any type of electrical conductor. The capacitor plate 130 can be formed, for example, from a solid plate, a thin film, foil, a conductive mesh, a conductive grid, one or more wires, or any other electrically conductive material. Although the capacitor plates 130 in FIG. 2 are shown as being rectangular in shape, any shape can be used for the conductive plates 130.

The CSA assembly 105 preferably includes an AC shield layer 150 that minimizes parasitic capacitance and noise coupling, and preferably includes a common potential layer 160 that capacitively couples a human body 170 to the common potential of the CSA assembly 105 and provides a common reference for the capacitance measurements. Although the common potential layer 160 is shown as a layer below the CSA 110, it should be appreciated that other configurations can be used while still falling within the scope of the present invention. For example, the AC shield layer 150 and common potential layer 160 could be positioned on a side surface 155 of the CSA 110. Further, although FIG. 1 shows that the common potential layer 160 is coupled to the human body 170 via capacitive coupling, the common potential layer 160 may also be electrically coupled to the human body 170 with a direct electrical connection using a conductive material.

Although a common potential layer 160 is preferably used, it is not required. Without a common potential layer 160, the capacitance between adjacent capacitor plates 130 changes and can be measured, with each capacitor plate 130 having distinct electrical potentials. The capacitance between adjacent plates 130 changes when a body part moves in proximity to the capacitor plates 130. The capacitance measurements may be made one at a time, but they are preferably made simultaneously in sets of two or more to cancel electrical noise and to obtain multiple measurements with temporal coincidence.

The capacitor plates 130, AC shield 150 and common potential plane 160 are preferably made from conductive textile that can be integrated into or mounted on the supporting structure 40 (not shown). The term “conductive textile” refers generally to a fabric that can conduct electricity. Conductive textiles can be made with metal strands that are woven into the construction of the textile. Conductive textiles can also be made with conductive fibers which, for example, may consist of a non-conductive or less conductive substrate that is either coated or embedded with electrically conductive elements, such as carbon, nickel, copper, gold, silver or titanium.

Examples of non-conductive or less conductive substrates include cotton, polyester, nylon stainless steel. Some examples of commercially available conductive textiles include those manufactured by Shieldex (low resistance, 4 Ohms per centimeter), MedTex (has various varieties such as E 130 DS (13 Ohm per 20 cm) and P 180 OS), LessEmf (stretchable conductive fabric that has a resistance of 13 Ohm per 20 cms), and Zelt Conductive fabric (0.4 Ohms per 20 cm).

The CSA 110 is preferably made by cutting patches of different shapes from the conductive textile and sewing them on to the fabric. Connections from the conductive patch (capacitor plate 130) is routed to a capacitance measurement circuit using conductive wires. These conductive wires are suitably ordinary threads coated with silver.

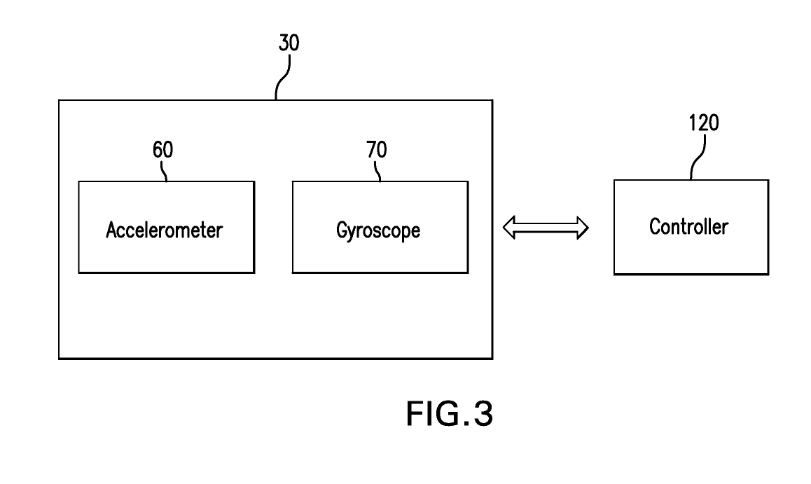

FIG. 3 is a schematic diagram of one preferred embodiment of the inertial sensing system 30. As discussed above, the inertial sensing system 30 can include any number of inertial sensors. However, in a preferred embodiment the inertial sensing system 30 preferably includes an accelerometer 60 and a gyroscope 70 for detecting motion and position. As discussed above, the inertial sensing system 30 communicates with controller 120 via connections 145, which can be wired connections, wireless connections, wireless inductive connections and/or capacitive connections using systems and methods well known in the art.

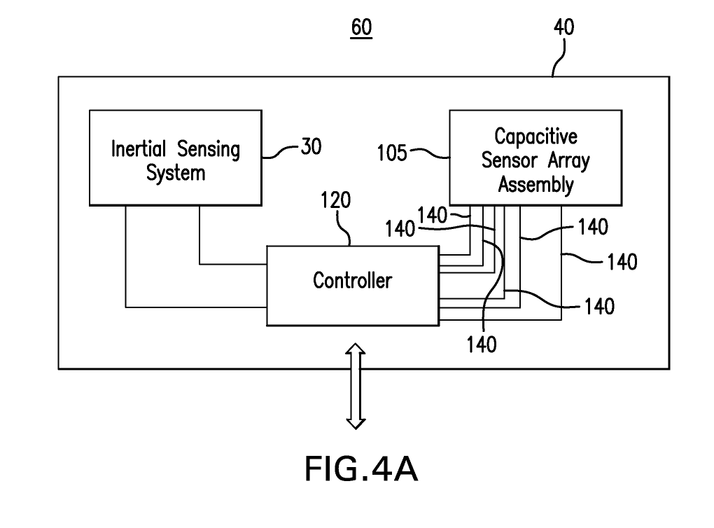

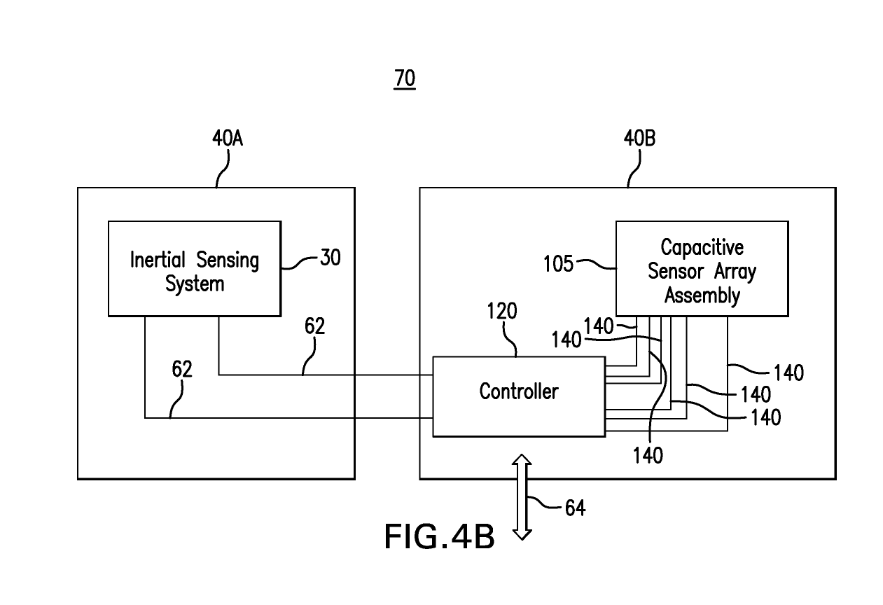

Although in the MSMS embodiments 10 and 50 shown in FIGS. 1A and 1B, respectively, the controller 120 is shown separate from the multi-sensor assembly 20, the controller 120 can also be mounted on or integrated with support structure 40, 40A or 40B, as shown in FIGS. 4A and 4B, which are schematic diagrams of MSMS embodiments 60 and 70, respectively, in accordance with other preferred embodiments of the present invention.

In the FIG. 4A embodiment 60, the inertial sensing system 30, CSA assembly 105 and controller 120 are all mounted on or integrated into support structure 40. Conductive wires 140 carry signals from the CSA assembly 105 to the controller 120. Conductive wires 62 carry signals from inertial sensing system 30 to the controller 120.

Connection 64 is used to communicate with the controller 120. Connection 64 can be a wired connection, a wireless connection, a wireless inductive connection and/or a capacitive connection using systems and methods well known in the art. However, connection 64 is preferably a wireless connection, which is suitably a Bluetooth radio link, an IEEE standards-based radio frequency link (WiFi), or any other type of radio frequency link.

As discussed above, multiple support structures 40 may be used while still falling within the scope of the present invention. In the embodiment 70 of FIG. 4B, inertial sensing system 30 is mounted on or integrated into support structure 40A, and CSA assembly 105 and controller 120 are mounted on or integrated into support structure 40B.

Although two support structures are shown in the embodiments of FIGS. 1B and 4A, it should be appreciated that any number of support structures may be used while still falling within the scope of the present invention. Further, any arrangement of the inertial sensing system 30, the CSA assembly 105 and the controller 120 among the one or more support structures 40, 40A and 40B may be implemented while still falling within the scope of the present invention. For example, in the embodiment 70 of FIG. 4B, which utilizes two support structures 40A and 40B, the controller 120 could be mounted on or integrated into support structure 40A, along with inertial sensing system 30. As another example, the inertial sensing system 30 and the CSA assembly 105 could be positioned on one support structure 40A or 40B, and the controller 120 could be positioned on the other support structure 40A or 40B.

Although six conductive wires 140 are shown in connection with CSA assembly 105 and two conductive wires 62 are shown in connection with inertial sensing system 30, it should be appreciated that any number of conductive wires can be used, depending on the number of capacitor plates 130 (not shown in FIGS. 4A and 4B) and the number of inertial sensors used in the inertial sensing system 30.

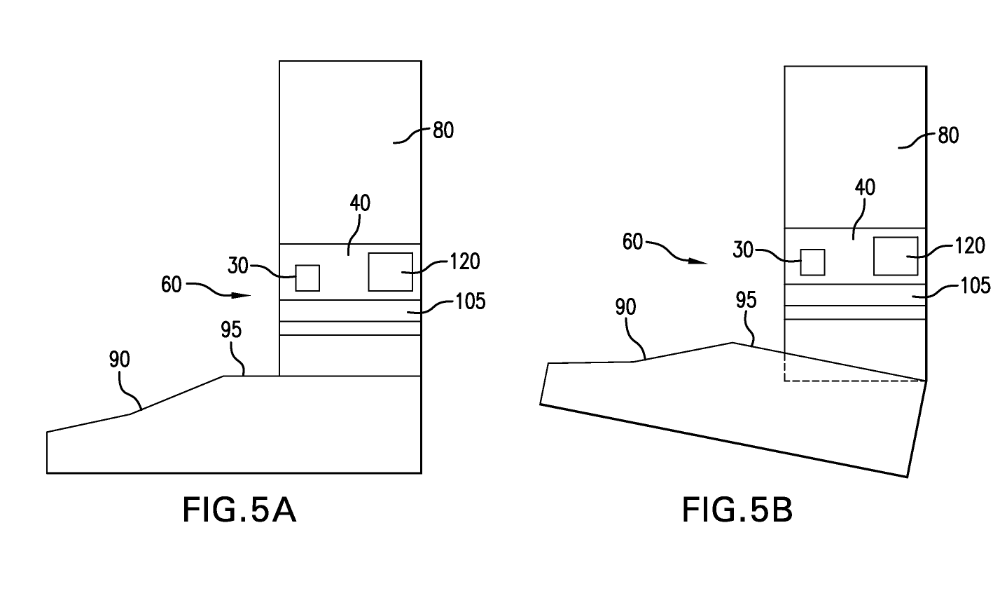

FIGS. 5A and 5B are schematic diagrams illustrating the MSMS embodiment 60 attached to a first body part 80 for measuring movement of the first body part 80 and movement of a second body part 90. In the example illustrated in FIGS. 5A and 5B, the first body part 80 is a leg and the second body part 90 is an appendage of the first body part (a foot). However, it should be appreciated that the MSMS can be used in in other applications in which one wants to distinctly measure the movement of a first object, as well as the movement of a second object relative to the first object (e.g., distinctly measure the movement of an arm and the movement of the hand relative to the arm or distinctly measure the movement of a leg and the movement of an arm as it sways past the leg while walking).

FIG. 5A represents the second body part 90 in a resting position and FIG. 5B represents the second body part 90 in a flexed position. The inertial sensing system 30 measures movement of the first body part 80, while the CSA assembly 105 measures movement of the second body part 90 via capacitive sensing, as will be explained in more detail below. As discussed above, the support structure 40 is preferably a flexible structure, suitably flexible fabric, that allows for the MSMS 60 to be wrapped around the first body part 80. As discussed above, examples of materials that can be used for the support structure 40 include, but are not limited to elastic bands and cotton ace bandages.

The flexible structure 40 is preferable wrapped around the first body part 80 and secured in place with any attachment mechanism known in the art, such as, for example, a Velcro® fastener (not shown). Alternatively, the support structure 40 can be made of an elastic band that fits around the first body part 80 and stays in place due to the force exerted by the elasticity of the band. In general, any method known in the art for securing a flexible support structure 40 to the first body part 80 may be used while still falling within the scope of the present invention.

One aspect of the present invention is the use of an array of capacitor plates 130. One of the advantages that a CSA 110 has over a single large capacitor plate is that taking differentials between capacitor plates 130 helps minimize noise due to stray movements in the vicinity of the plates 130. Also, the use of a CSA 110 instead of a single capacitor plate provides directional selectivity. Movement from the direction of the second body part 90 can be uniquely detected and distinguished from movement of other body parts or foreign bodies or electromagnetic interference by comparing the capacitive readings among the various plates 130.

As the joint of the second body part 90 is flexed, capacitance increases as the second body part 90 is moved towards capacitor plates 130 and decreases as the second body part 90 is flexed away. The capacitor plates 130 that are closer to the second body part 90 exhibit larger variations in capacitance than capacitor plates 130 that are further from the second body part 90. Signals from the capacitive plates 130 of the CSA 110 are sent to the controller 120 and are used to determine movement and position information.

As the second body part 90 is flexed, residual movement on the first body part 80 will be detected by the inertial sensor 30, but the movement detected is significantly larger when the first body part 80 itself is moved. By correlating the readings from the inertial sensor 30 and readings from the capacitive plates 130, one can determine with high confidence if the first body part 80 moved, if the second body part 90 moved, or if both the first body part 80 and the second body part 90 moved together, as defined by predetermined detection thresholds.

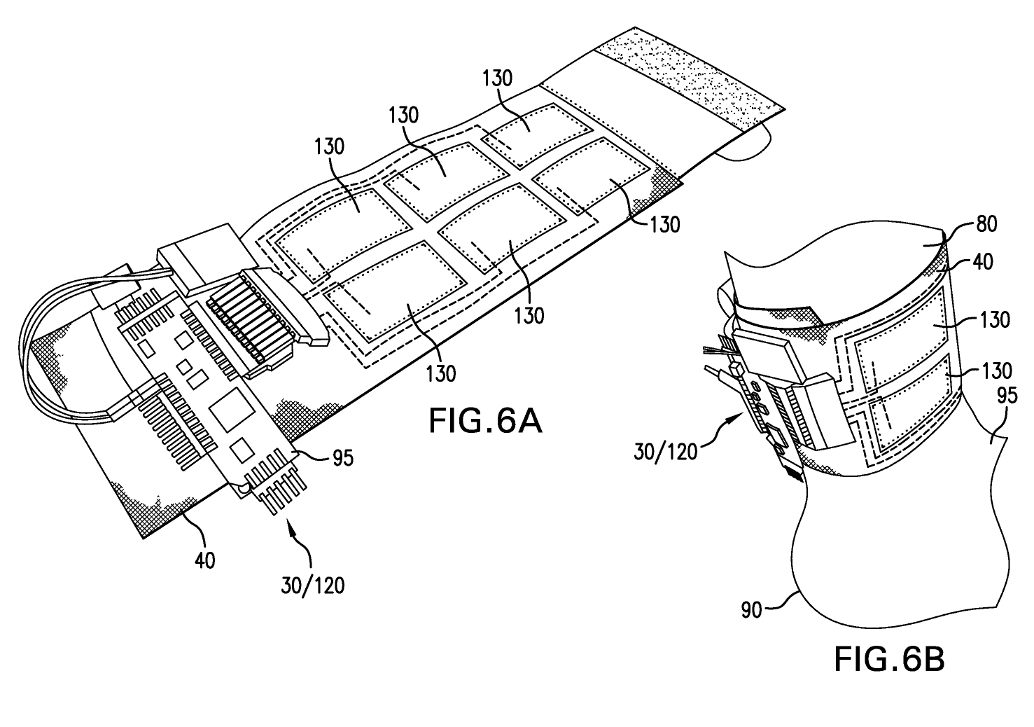

FIGS. 6A and 6B are perspective views showing the MSMS embodiment 60 in isolation (FIG. 6A) and wrapped around and secured to a first body part 80, in this example a leg (FIG. 6B). The embodiment shown in FIGS. 6A and 6B is wrapped around and secured around the first body part 80 using Velcro® fasteners (not shown). In this embodiment, the inertial sensing system 30 and controller 120 are mounted on a circuit board 95 that, in turn, is attached to supporting structure 40.

With capacitor plates 130 that have a size of 2 inches by 2 inches, the maximum distance between the top of the foot 95 and the CSA 110 that will cause a measurable change in capacitance is approximately 3 inches. The maximum distance can be adjusted by varying the size and shape of the capacitor plates 130.

Data accumulated by controller 120 regarding foot flexion, leg movements and leg positions is preferably used to calculate PLM/PLMS and GLM/GLMS indices and features, including how movement features relate to medical conditions. This is suitably accomplished using a backend processor (not shown). The data can also be used to provide sleep indices, activity visualizations and logs for a user and/or a detailed report with appropriate added analytics/normative data for a physician.

The controller 120 can be implemented with any type of processing device, such as a special purpose computer, a server, a tablet computer, a smartphone, a programmed microprocessor or microcontroller and peripheral integrated circuit elements, ASICs or other integrated circuits, hardwired electronic or logic circuits such as discrete element circuits, programmable logic devices such as FPGA, PLD, PLA or PAL or the like. In general, any device on which a finite state machine capable of running the programs and/or applications used to implement the systems and methods described herein can be used as the controller 120.

System Evaluation

A major challenge for an ankle-worn MSMS is to assess foot flexion and leg activity for different sleep body and leg positions and for ambient movements in the nearby surrounding. For instance, users at home may cross legs or sleep with one leg on top of the other and with a pillow in between. The present invention addresses this challenge by analyzing data from a CSA 110, an accelerometer 60 and, optionally, a gyroscope 70. The CSA 110 provides multi-vantage measurements of body movements. The amplitude difference of the measured capacitance at different capacitance plates 130, in conjunction with data from the accelerometer 60 and gyroscope 70, can be used to determine the orientation of the first body part 80 (leg) with respect to the bed, while analog differentials between the capacitance values can be used to filter noise due to ambient movements and detect second body part 90 (foot) movement relative to current surroundings.

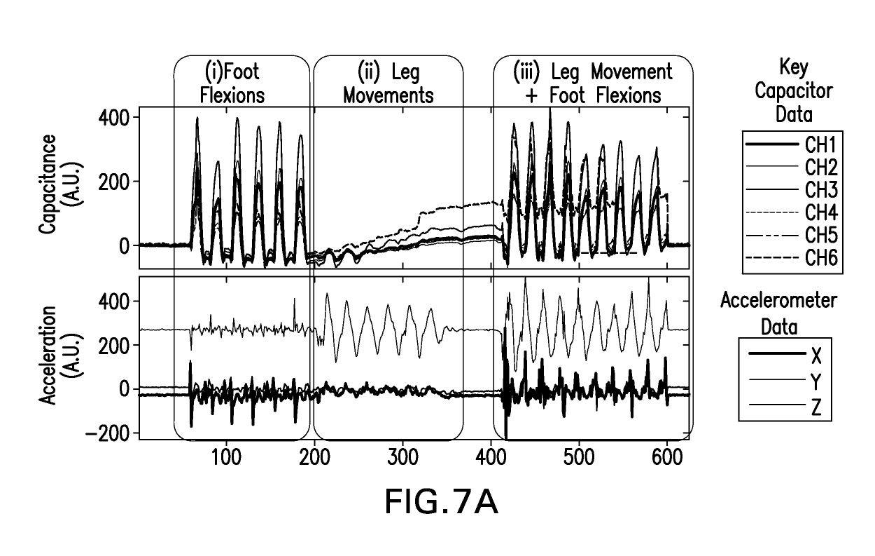

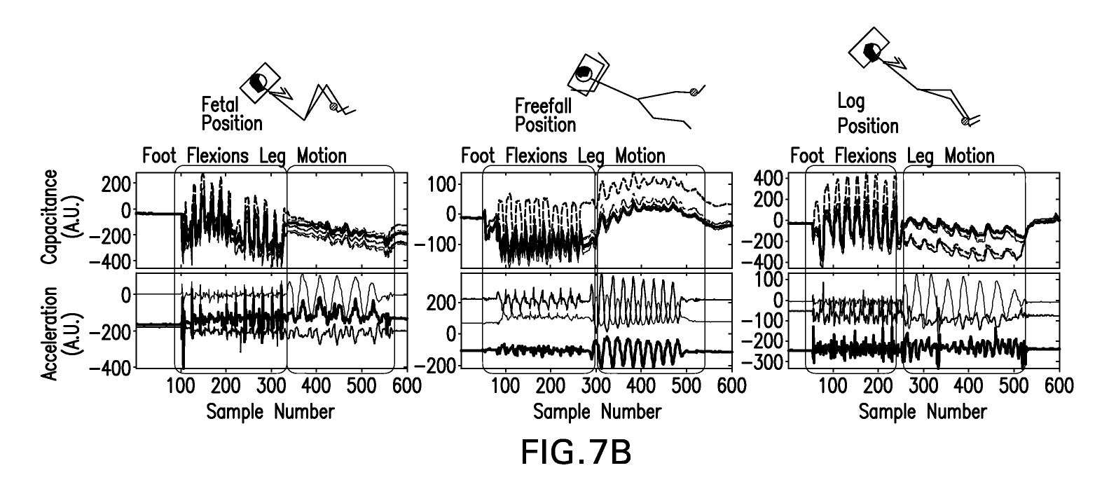

Initial data from the MSMS demonstrates that a combination of a CSA 110 and an accelerometer 60 can be used to distinguish foot flexion from leg movement, as shown in FIGS. 7A and 7B. In this study, the sensor MSMS was wrapped around the ankle of a subject and the subject performed 3 types of leg movements: (i) foot flexion with no leg movements; (ii) leg movement with no foot flexion; and (iii) leg movement and foot flexion. The results are shown in FIG. 7A, which are graphs showing the capacitance measured by the six capacitor plates 130 of the CSA 110, as well as the acceleration measured by the accelerometer 60. The graphs shows that the CSA 110 is able to reliably capture foot flexion, while the accelerometer 60 reliably captures leg movement without using sensors at multiple sites.

The MSMS can accommodate various sleep-leg postures. A preliminary review of videotapes and web-based presentations revealed six major sleep positions, including feet crossed and one foot on top of the other. Initial data from a few sleep positions is shown in FIG. 7B, which are graphs showing the capacitance measured by the six capacitor plates 130 of the CSA 110, as well as the acceleration measured by the accelerometer 60 for three different sleep positions: (i) fetal position; (ii) freefall position; and (iii) log position.

Though characteristic responses vary for each position, the CSA 110 and accelerometer 60 provides increased observability and demonstrates consistent distinction between responses from general leg movement versus foot flexions. In the trial that produced the data shown in FIG. 7B, video capture revealed that the subject did not completely isolate the two types of movements, which can be seen in the data shown in FIG. 7B.

The foregoing embodiments and advantages are merely exemplary, and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. Various changes may be made without departing from the spirit and scope of the invention, as defined in the following claims (after the Appendix below).

### APPENDIX

1. Trotti, L. M., et al., Correlates of PLMs variability over multiple nights and impact upon RLS diagnosis. Sleep Med, 2009. 10: p. 668-671.
2. Picchietti, M. A., et al., Children show individual night-to-night variability of periodic limb movements in sleep. Sleep, 2009. 32(4): p. 530-5.
3. Terrill, P. I., et al., Measuring leg movements during sleep using accelerometry: comparison with EMG and piezo-electric scored events. Conf Proc IEEE Eng Med Biol Soc, 2013. 2013: p. 6862-5.
4. Montplaisir, J., et al., Clinical, polysomnographic, and genetic characteristics of restless legs syndrome: a study of 133 patients diagnosed with new standard criteria. Mov Disord, 1997. 12(1): p. 61-5.
5. Montplaisir, J., et al., Periodic leg movements are not more prevalent in insomnia or hypersomnia but are specifically associated with sleep disorders involving a dopaminergic impairment. Sleep Med, 2000. 1(2): p. 163-167.
6. Pennestri, M. H., et al., PLMS and PLMW in healthy subjects as a function of age: prevalence and interval distribution. Sleep, 2006. 29(9): p. 1183-7.
7. Nicolas, A., P. Lesperance, and J. Montplaisir, Is excessive daytime sleepiness with periodic leg movements during sleep a specific diagnostic category? Eur Neurol, 1998. 40(1): p. 22-6.
8. Siddiqui, F., et al., Rise of blood pressure with periodic limb movements in sleep and wakefulness. Clinical Neurophysiology, 2007. 118(9): p. 1923-30.
9. Pennestri, M. H., et al., Nocturnal blood pressure changes in patients with restless legs syndrome. Neurology, 2007. 68(15): p. 1213-8.
10. Koo, B. B., et al., Association of incident cardiovascular disease with periodic limb movements during sleep in older men: outcomes of sleep disorders in older men (MrOS) study. Circulation, 2011. 124(11): p. 1223-31.
11. Mirza, M., et al., Frequent periodic leg movement during sleep is associated with left ventricular hypertrophy and adverse cardiovascular outcomes. J Am Soc Echocardiogr, 2013. 26(7): p. 783-90.
12. Giannaki, C. D., et al., Periodic limb movements in sleep contribute to further cardiac structure abnormalities in hemodialysis patients with restless legs syndrome. J Clin Sleep Med, 2013. 9(2): p. 147-53.
13. Nannapaneni, S. and K. Ramar, Periodic limb movements during sleep and their effect on the cardiovascular system: is there a final answer? Sleep Medicine, 2014. 15(4): p. 379-384.
14. Garcia-Borreguero, D., et al., Correlation between rating scales and sleep laboratory measurements in restless legs syndrome. Sleep Medicine, 2004. 5(6): p. 561-565.
15. Allen, R. P. and C. J. Earley, Validation of the Johns Hopkins restless legs severity scale. Sleep Med, 2001. 2: p. 239-242.
16. Allen, R. P., et al., A double-blind, placebo controlled study of the treatment of periodic limb movements in sleep using carbidopa/levodopa and propoxyphene. Sleep, 1993. 16: p. 717-723.
17. Kaplan, P. W., et al., A double-blind, placebo-controlled study of the treatment of periodic limb movements in sleep using carbidopa/levodopa and propoxyphene. Sleep, 1993. 16(8): p. 717-723.
18. Partinen, M., et al., Efficacy and safety of pramipexole in idiopathic restless legs syndrome: a polysomnographic dose-finding study—the PRELUDE study. Sleep Med, 2006. 7(5): p. 407-17.
19. Michaud, M., et al., Sleep laboratory diagnosis of restless legs syndrome. Eur Neurol, 2002. 48(2): p. 108-13.
20. Fulda, S., et al., P6. Leg movements during sleep in patients with obstructive sleep apnea. Clinical Neurophysiology, 2012. 123(10): p. e104.
21. Manconi, M., et al., Time structure analysis of leg movements during sleep in REM sleep behavior disorder. Sleep, 2007. 30(12): p. 1779-85.
22. Winkelman, J., High rates of periodic leg movements of sleep in REM-sleep behavior disorder raise more questions about the relationship between sleep-related movement disorders. Article reviewed: M. L. Fantini, M. Michaud, N Gosselin, G. Lavigne, J. Monlaisir, Periodic leg movements in REM sleep behavior disorder and related autonomic and EEG activation, Neurology, 59 (2002) 1889-1894. Sleep Med, 2003. 4(3): p. 261-2.
23. Moore, H. t., et al., Periodic Leg Movements during Sleep Are Associated with Polymorphisms in BTBD9, TOX3/BC034767, MEIS1, MAP2K5/SKOR1, and PTPRD. Sleep, 2014. 37 (9).
24. Stefansson, H., et al., A Genetic Risk Factor for Periodic Limb Movements in Sleep. N Engl J Med, 2007. 357(7): p. 639-647.
25. Birinyi, P. V., et al., Undiagnosed individuals with first-degree relatives with restless legs syndrome have increased periodic limb movements. Sleep Med, 2006. 7(6): p. 480-5.
26. Marcus, C. L., et al., Prevalence of periodic limb movements during sleep in normal children. Sleep, 2014. 37(8): p. 1349-52.
27. Sadeh, A., L. Pergamin, and Y. Bar-Haim, Sleep in children with attention-deficit hyperactivity disorder: a meta-analysis of polysomnographic studies. Sleep Med Rev, 2006. 10(6): p. 381-98.
28. Picchietti, D. L. and H. E. Stevens, Early manifestations of restless legs syndrome in childhood and adolescence. Sleep Med, 2008. 9(7): p. 770-81.
29. Picchietti, D., et al., Restless legs syndrome: prevalence and impact in children and adolescents—the Peds REST study. Pediatrics, 2007. 120(2): p. 253-66.
30. Simakajornboon, N., et al., Periodic limb movements in sleep and iron status in children. Sleep, 2003. 26(6): p. 735-8.
31. Tilma, J., et al., Early childhood-onset restless legs syndrome: symptoms and effect of oral iron treatment. Acta Paediatr, 2013. 102(5): p. e221-6.
32. Kobayashi, M., et al., The validity of the PAM-RL device for evaluating periodic limb movements in sleep and an investigation on night-to-night variability of periodic limb movements during sleep in patients with restless legs syndrome or periodic limb movement disorder using this system. Sleep Med, 2014. 15(1): p. 138-43.
33. Sforza, E., M. Johannes, and B. Claudio, The PAM-RL ambulatory device for detection of periodic leg movements: a validation study. Sleep Med, 2005. 6(5): p. 407-13.

What is claimed is:

1. A multi-sensor measurement system, comprising:
at least one support structure;
a capacitive sensor array disposed on the at least one support structure, wherein the capacitive sensor array comprises at least two conductive plates; and
at least one inertial sensor disposed on the at least one support structure.

2. The system of claim 1, wherein the at least one support structure is flexible.

3. The system of claim 2, wherein the at least one support structure comprises flexible fabric and the at least two conductive plates are flexible.

4. The system of claim 3, wherein the at least two conductive plates are formed with conductive textile.

5. The system of claim 4, wherein the capacitor sensor array is integrated with the flexible fabric that comprises the at least one support structure.

6. The system of claim 2, wherein the support structure is adapted to be wrapped around a body part.

7. The system of claim 1, wherein the at least one inertial sensor comprises an accelerometer.

8. The system of claim 7, wherein the at least one inertial sensor comprises an accelerometer and a gyroscope.

9. The system of claim 1, wherein the at least two capacitor plates comprises six capacitor plates.

10. The system of claim 1, further comprising a controller in communication with the capacitive sensor array and the at least one inertial sensor.

11. The system of claim 10, wherein the controller determines movement and position information of an object in proximity to the capacitive sensor array based on capacitance signals received from the capacitive sensor array.

12. The system of claim 10, wherein the controller is disposed on the at least one support structure.

13. The system of claim 12, wherein the controller and the at least one inertial sensor are mounted on a common circuit board.

14. The system of claim 1, further comprising a common potential layer spaced apart from the at least two conductive plates.

15. The system of claim 14, further comprising an AC shield layer positioned between the at least two conductive plates and the common potential layer.

16. The system of claim 1, wherein the at least one support structure comprises a first support structure and a second support structure, and wherein the capacitive sensor array is disposed on the first support structure and the at least one inertial sensor is disposed on the second support structure.

17. The system of claim 10, wherein the controller is in communication with the capacitive sensor array and the at least one inertial sensor via a wireless connection.

18. A system for detecting movement of a first body part and movement of a second body part with respect to the first body part, comprising:
at least one flexible support structure, wherein the at least one flexible support structure is adapted to be wrapped around the first body part;
a capacitive sensor array disposed on the at least one flexible support structure, wherein the capacitive sensor array comprises at least two flexible conductive plates; and
at least one inertial sensor disposed on the at least one flexible support structure.

19. The system of claim 18, wherein the at least one flexible support structure comprises flexible fabric.

20. The system of claim 19, wherein the at least two flexible conductive plates are formed with conductive textile.

21. The system of claim 20, wherein the capacitor sensor array is integrated with the flexible fabric that comprises the at least one support structure.

22. The system of claim 18, wherein the at least one inertial sensor comprises an accelerometer.

23. The system of claim 22, wherein the at least one inertial sensor comprises an accelerometer and a gyroscope.

24. The system of claim 18, wherein the at least two flexible capacitor plates comprises six flexible capacitor plates.

25. The system of claim 18, further comprising a controller in communication with the capacitive sensor array and the at least one inertial sensor.

26. The system of claim 25, wherein the controller determines movement and position information of a second object in proximity to the capacitive sensor array based on capacitance signals received from the capacitive sensor array.

27. The system of claim 18, wherein the flexible support structure is adapted to be wrapped around a leg.

28. The system of claim 18, further comprising a common potential layer spaced apart from the at least two conductive plates.

29. The system of claim 28, further comprising an AC shield layer positioned between the at least two conductive plates and the common potential layer.

30. A method of detecting movement of a first body part and movement of a second body part relative to the first body part, comprising the steps of:
detecting movement of the first body part based on signals from at least one inertial sensor; and
detecting movement of the second body part relative to the first body part based on signals from a capacitive sensor array.

31. The method of claim 30, wherein the movement of the first body part is detected based on signals from an accelerometer.

32. The method of claim 30, wherein movement of the second body part is detected based on a change in capacitance caused by movement of the second body part.

33. The method of claim 30, further comprising the step of determining position information for the first body part based on signals from a gyroscope and accelerometer.

34. The method of claim 30, wherein the first body part comprises a leg and the second body part comprises a foot.

35. The method of claim 34, further comprising the step of calculating periodic leg movements (PLM) and general leg movements (GLM) based on the detected movements of the leg and foot.
